(19) United States
(12) Patent Application Publication
(10) Pub. No.: US 2002/0037534 A1
(43) Pub. Date: Mar. 28, 2002

(54) Combinatorial preparation of a substance library with removal of a zwitterionic compound by precipitation

(76) Inventors: Lutz F. Tietze, Gottingen (DE); Holger Evers, Madison, WI (US); Enno Topken, Gottingen (DE)

Correspondence Address: Herbert B. Keil, KEIL & WEINKAUF, 1101 Connecticut Ave., N.W., Washington, DC, 20036, US

(21) Appl. No.: 09/949,571

(22) Filed: Sep. 11, 2001

(30) Foreign Application Priority Data
Sep. 13, 2000 (DE) 10045530.1

(51) Int. Cl. G01N 33/53; C07C 229/26

(52) U.S. Cl. 435/7.1; 562/443; 562/567

(57) ABSTRACT

A process for the combinatorial preparation of a substance library from n different compounds in an array of n different reaction solutions at n spatially separate reaction sites, in which at each of the n reaction sites

(a) from in each case m reactants, in one reaction or reaction sequence, a zwitterionic compound is obtained dissolved in a polar solvent,

(b) the zwitterionic compound is precipitated as a solid by addition of a nonpolar solvent, isolated and then obtained in pure form,

(c) if appropriate the zwitterionic compound is further reacted, with each of the n reaction solutions differing from each of the n−1 other reaction solutions in at least one of the m reactants,

is described.

The invention relates to a process for preparing a substance library from n different compounds.

Combinatorial synthesis is currently carried out both in the liquid and solid phases. Both processes have disadvantages. In solid phase synthesis, two steps must be additionally carried out, that is to say the linking to and cleavage from a polymeric support. In addition, many reactions are not currently possible, or are possible only with limitations, in the solid phase. In the case of liquid-phase synthesis, the removal of excess reagents and purifying the reaction products is problematic.

It is an object of the present invention to provide a process for the combinatorial synthesis of a substance library which combines the advantages of the conventional solid-phase and liquid-phase syntheses, but substantially avoids their disadvantages.

We have found that this object is achieved by a process for the combinatorial preparation of a substance library from n different compounds in an array of n different reaction solutions at n spatially separate reaction sites, in which at each of the n reaction sites

(a) from in each case m reactants, in one reaction or reaction sequence, a zwitterionic compound is obtained dissolved in a polar solvent,

(b) the zwitterionic compound is precipitated as a solid by addition of a nonpolar solvent, isolated and then obtained in pure form,

(c) if appropriate the zwitterionic compound is further reacted, with each of the n reaction solutions differing from each of the n−1 other reaction solutions in at least one of the m reactants.

In the inventive process, a zwitterionic compound (a betaine) is formed as intermediate or end product which is soluble in aqueous, aqueous-organic or polar organic media, but is insoluble in nonpolar media. As a result, the zwitterionic compound formed in the course of a reaction or reaction sequence can be removed from the reaction mixture without problem, by precipitating it as a solid from solution in a polar solvent by adding a nonpolar organic solvent and isolating it.

Suitable polar solvents are all solvents in which the zwitterionic compounds are soluble. Examples are water and alcohols such as methanol and ethanol or their mixtures. Suitable nonpolar solvents are those which are at least partially miscible with the polar solvents. One example is diethyl ether. By adding the nonpolar solvent to the solution of the zwitterionic compound, a solvent mixture of low polarity is obtained, whereupon the zwitterionic compound precipitates out. The precipitated solid is obtained as a result in high purity.

The zwitterionic compound is obtained dissolved in a polar solvent. The zwitterionic compound can already be formed in the polar solvent, or the previously formed compound can be dissolved in the polar solvent in a workup step.

In one embodiment of the inventive process, as m reactants, a protected aminoaldehyde of the general formula 1, 2 or 3 is reacted with an enol ether of the general formula 5 and a 1,3-dicarbonyl compound 4 in the reaction sequence below to form a betaine of the general formula 6, 7 or 8 as zwitterionic compound:

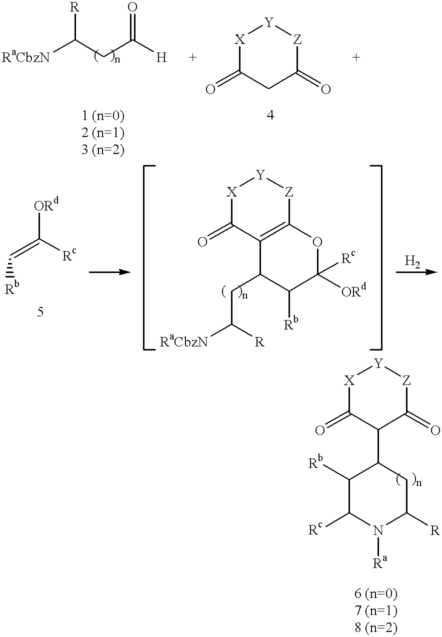

Cbz is a benzyloxycarbonyl group and R^{d} is a benzyl or trimethylsilyl group here.

Condensation of the protected aminoaldehyde 1, 2 or 3 with the 1,3-dicarbonyl compound 4 and subsequent hetero Diels-Alder reaction of the condensation product formed with the enolether 5 produces an acetal as intermediate. The acetal is then converted into free aldehyde, or if R^{c}≢H, into the free ketone, by hydrogenolytic removal of the benzyl protecting group. Hydrogenolytic removal of the benzyloxycarbonyl group furthermore forms a free amine function. By intramolecular condensation of the aldehyde with the amine, a cyclic enamine/imine is formed in situ which is reduced under the hydrogenolysis conditions to give the corresponding saturated nitrogen heterocycle 6, 7 or 8.

The protected aminoaldehyde 1, 2 or 3 is preferably condensed with the 1,3-dicarbonyl compound 5 in the presence of an acid or basic catalyst such as ethylenediammonium diacetate and/or a dehydrating agent such as trialkyl orthoformate. Operations are preferably carried out in toluene as solvent and under a protective gas atmosphere.

Hydrogenolysis can be carried out on any suitable hydrogenation catalyst, for example palladium on activated carbon. The process is preferably carried out in methanol as solvent, the betaine 6, 7 or 8 being obtained in methanol as polar solvent. Preferably, the betaine is precipitated by adding diethyl ether as nonpolar solvent. The betaine solution can be concentrated by distilling off methanol before the precipitation step.

The betaines 6, 7 and 8 are isolated at a purity of preferably 80 to 100 mol %.

Suitable compounds 1 to 5 are, for example, those which contain pharmacophore groups, such as barbituric acids and hydroxycoumarines, and other 1,3-dicarbonyl compounds, which can be reacted with aminoaldehydes, which can be prepared from natural and synthetic amino acids and amino alcohols, and highly varied benzylenol ethers.

Preferred protected aminoaldehydes are the alpha-, beta- and gamma-aminoaldehydes of the general formulae 1a to 1e:

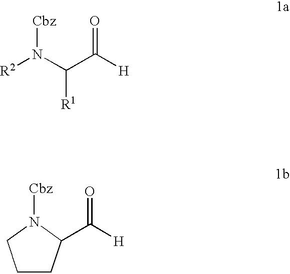

where

R¹=H, alkyl, CH₂OH, CH₂SH,

R²=H, alkyl and

alkyl=Me, Et, n-Pr, i-Pr, n-Bu, sec-Bu, i-Bu or pentyl

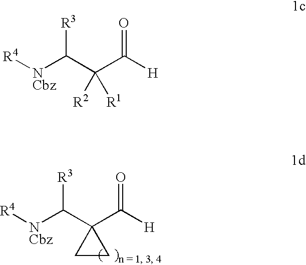

where

R¹, R² are identical or different and are H alkyl

R³, R⁴ are identical or different and are H, alkyl and alkyl=Me, Et, n-Pr, iPr, n-Bu, sec-Bu, iBu or pentyl

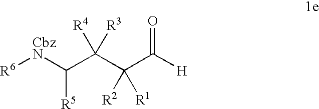

where

R¹, R², R³, R⁴ are identical or different and are H, alkyl

R⁵, R⁶ are identical or different and are H, alkyl,

and alkyl =Me, Et, n-Pr, n-Bu, sec-Bu, iBu or pentyl.

Preferred enol ethers are those of the general formulae 2a to 2d:

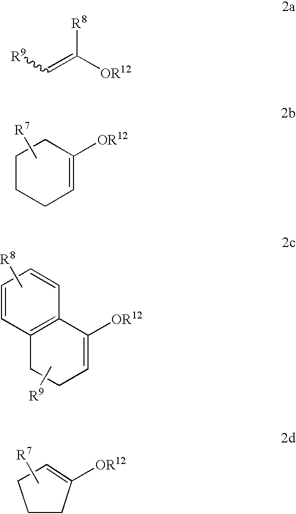

where

R⁷, R⁸, R⁹ are identical or different and are H, alkyl, Bn, Ph-C₂H₄, CH₂OR, CH₂SR, CH₂NHAc, CH₂CH₂OR, CH₂CH₂SR, CH₂CH₂NHAc and

R⁹ is additionally Ph,

R¹² is benzyl or —Si(CH₃)₃,

and alkyl=Me, Et, n-Pr, iPr, n-Bu, sec-Bu, iBu or pentyl.

Preferred 1,3-dicarbonyl compounds are those of the general formulae 3a and 3b:

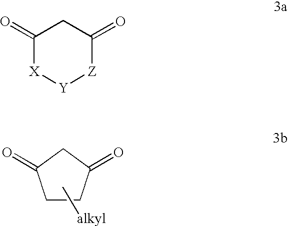

where

X, Y are identical or different and are NH, N-alkyl, CH₂, CMe₂, O, S and

Y is CH₂, CMe₂, C═O, C═S, or X—Y—Z is NH—CR¹⁰═CR¹¹, N-alkyl-CR¹⁰═CR¹¹, O—CR¹⁰═CR¹¹, N-alkyl-CHR¹—CHR², where R¹⁰ and R¹¹ are identical or different and are alkyl, unsubstituted or with OH, O-alkyl, NH₂, NHalkyl or N(alkyl)₂ substituted phenyl, unsubstituted or with OH, O-alkyl, NH₂, NHalkyl or N(alkyl)₂ substituted 1,3-butadiene-1,3-dienyl, where in the last case R¹⁰ and R¹¹ are part of the same ring system, and alkyl=Me, Et, n-Pr, iPr, n-Bu, sec-Bu, i-Bu or pentyl.

In the above, Me=methyl, Et=ethyl, Pr=propyl, Bu=butyl, Ph=phenyl and Bn=benzyl.

Some example reactions are listed below.

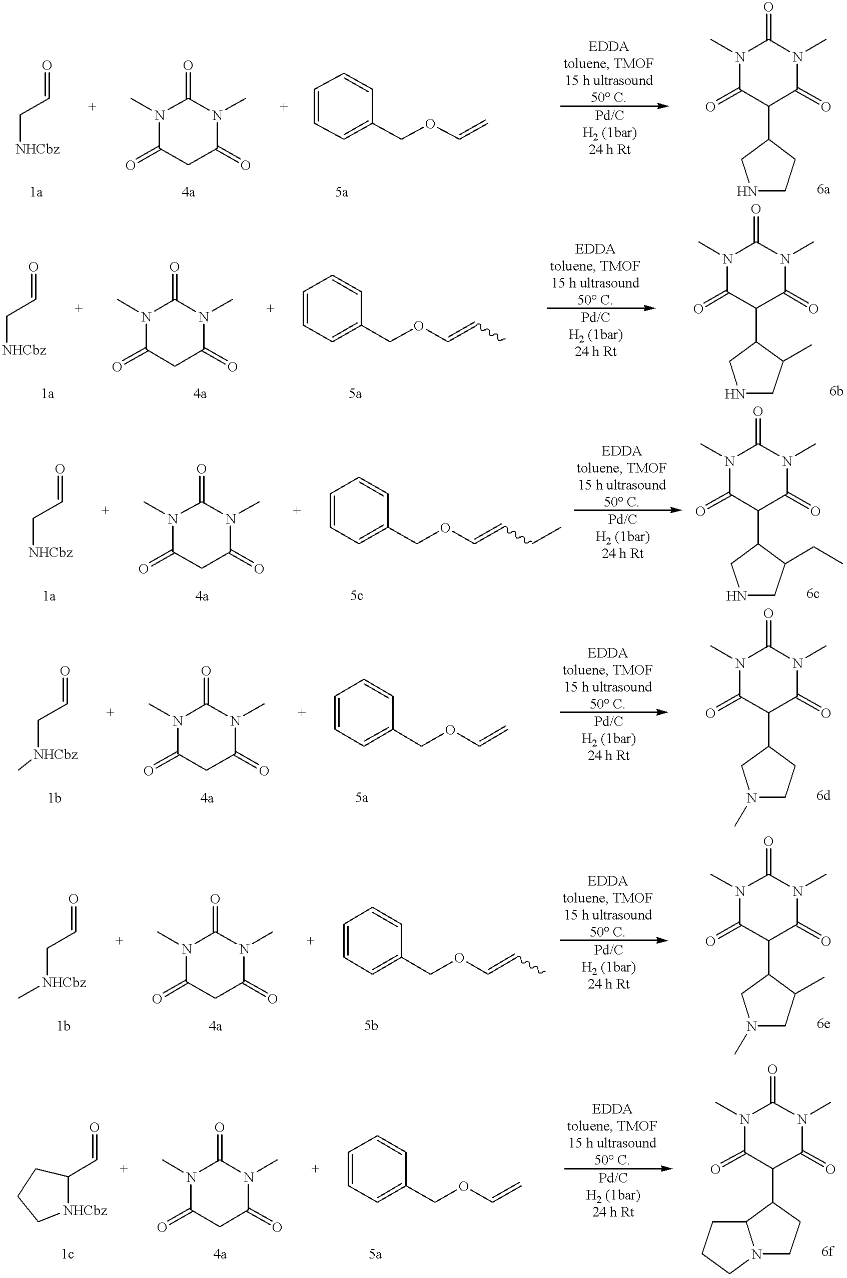

By combining the aminoaldehydes 1, 2 or 3 with the dicarbonyl compounds 5 and the enolethers 4, a very large number of different betaines 6, 7 and 8 can be obtained.

The zwitterionic compounds isolated can be further reacted.

The reactions are carried out in n different reaction mixtures at n spatially separated reaction sites, where n is at least 2. Per reaction mixture, at least one of the m reactants is varied, so that each of the n reaction mixtures differs from the n−1 remaining reaction mixtures and at least one of the m reactants. For example, the combination of 10 different aminoaldehydes 1, 2 or 3 with 10 different enolethers 5 and 10 different 1,3-dicarbonyl compounds 4 gives in total 1000 different reaction mixtures. The n reactions can be carried out in parallel and automated, in which case customary automated synthesis systems can be used. In the case of automated operations, n can be very large, for example up to 100, 1000 and 10 000. Certain substeps, for example the reaction steps, can also be carried out in an automated manner and other steps, for example the workup steps, can be carried out manually. In each case the problem-free removal of the zwitterionic compound from the reaction mixture as a solid by precipitation and isolation makes possible considerable savings in time, so that even with manual or semi-automated operations, a large number of reactions, for example 10 or 100 reactions, can be carried out in parallel.

The resulting compounds can be tested in the suitable test systems, for example, for their pharmacological activity and/or for their efficacy as crop protection agents.

The invention is described in more detail by the examples below.

General operating procedure

GOP: Domino-Knoevenagel-hetero-Diels-Alder reaction with protected aminoaldehydes

1.0 equivalent of the Cbz-protected aminoaldehyde, 1.0 equivalent of the 1,3-dicarbonyl component, 4.0 equivalents of enolether and toluene (4 ml/mmol), trimethyl orthoformate (0.8 ml/mmol) and a few crystals of ethylenediammonium diacetate (EDDA) were combined under an argon atmosphere in a 10 ml pressurized flask. The reaction mixture was sonicated in an ultrasonic bath for 15 h at 50-60° C. (monitoring with TLC). After the reaction is complete, the solvent was removed on a rotary evaporator and the residue dissolved in methanol (4 ml/mmol). After addition of palladium on carbon (10%, 100 mg/mmol), the mixture was stirred for 24 h under a hydrogen atmosphere (TLC monitoring). The catalyst was then filtered off over a little celite, washed with methanol and the solvent was again taken off under reduced pressure. The residue was taken up in a little methanol and the product precipitated by slow addition of diethyl ether. The white amorphous precipitate was filtered off and washed with diethyl ether.

Products obtained

3-(1,3-Dimethyl-2, 4, 6-trioxohexahydropyrimidin-5-yl)-pyrrolidine (6a)

N-Cbz-Aminoacetaldehyde (24.1 mg, 125 μmol), N,N-dimethylbarbituric acid (19.5 mg, 125 μmol) and benzyl vinyl ether (134 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was>95%.

¹H-NMR (500 MHz, CD₃OD): δ=2.06-2.14, 2.18-2.25 (2m, 2H, 4-H), 3.14-3.26 (m, 8H, 2×NMe, 5-H), 3.41-3.47, 3.50-3.56 (2m, 2H, 2-H), 3.78-3.88 (m, 1H, 3-35 H).

¹³C-NMR (50 MHz, CD₃OD): δ=28.02, 34.30 (NMe, C-3), 30.22 (C-4), 88.14 (C-5′), 154.5 (C-2′), 165.1 (C-4′, C-6′). C₁₀H₁₅N₃O₃(225.1), HRMS: 225.1113.

3-(1,3-Dimethyl-2, 4, 6-trioxohexahydropyrmidin-5-yl)-4-methylpyrrolidine (6b)

N-Cbz-Aminoacetaldehyde (24.1 mg, 125 μmol), N,N-dimethylbarbituric acid (19.5 mg, 125 μmol) and benzyl prop-1-enyl ether (148 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was>95%.

MS (70 eV): m/z=239.1 (100) [M⁺], 156.1 (45) [C₆H₈N₂O₃], 83.0 (100) [M⁺—C₆H₈N₂O₃], 68.0 (73) [M⁺—C₆H₈N₂O₃- methyl].

C₁₁H₁₇N₃O₃(239.1), HRMS: 239.1269.

3-(1,3-Dimethyl-2, 4, 6-trioxohexahydropyrimidin-5-yl)-4-ethylpyrrolidine (6c)

N-Cbz-Aminoacetaldehyde (24.1 mg, 125 μmol), N,N-dimethylbarbituric acid (19.5 mg, 125 μmol) and benzyl but-1-enyl ether (162 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was>97%.

¹³C-NMR (50 MHz, CD₃OD): δ=12.75, 13.55 (C-2″), 23.45 (C-1″), 27.76, 27.78, 28.02, 28.23 (NMe), 40.64 (C-4), 44.93, 45.49 (C-3), 52.31 (C-2 or 5), 86.93, 87.65 (C-5′), 154.6, 154.7 (C-2′), 165.0, 165.2, 165.8 (C-4′, C-6′). MS (70 eV): m/z=253.2 (10) [M⁺], 156.1 (6) [C₆H₈N₂O₃], 97.1 (82) [M⁺—C₆H₈N₂O₃], 68.0 (100) [M⁺—C₆H₈N₂O₃- Et].

C₁₂H₁₉N₃O₃ (253.1), HRMS: 253.1426.

3-(1,3-Dimethyl-2, 4, 6-trioxohexahydropyrimidin-5-yl)-1-methylpyrrolidine (6d)

N-Cbz-Methylaminoacetaldehyde (25.9 mg, 125 μmol), N,N-dimethylbarbituric acid (19.5 mg, 125 μmol) and benzyl vinyl ether (134 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals.

¹³C-NMR (50 MHz, CD₃OD): δ=30.77 (C-4), 28.05, 33.95, 40.40 (C-3, CONMe, NMe), 57.42, 61.07 (C-2, C-5), 90.74 (C-5′), 154.5 (C-2′), 165.1 (C-4′, C-6′).

MS (70 eV): m/z=239.2 (20) [M⁺], 83.0 (100) [M⁺—C₆H₈N₂O₃].

C₁₁H₁₇N₃O₃ (239.2).

3-(1,3-Dimethyl-2, 4, 6-trioxohexahydropyrimidin-5-yl)-1,4-dimethylpyrrolidine (6e)

N-Cbz-Methylaminoacetaldehyde (25.9 mg, 125 μmol), N,N-dimethylbarbituric acid (19.5 mg, 125 μmol) and benzyl prop-1-enyl ether (148 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was>95%.

¹H-NMR (300 MHz, DMSO): δ=0.74, 0.95 (2d, J=6.5 Hz, 3H, 1″-H), 2.52-2.68 (m, 2H, 4-H), 2.71, 2.75 (2s, 3H, NMe), 2.70-3.84 (3m, 5H, 2-H, 3-H, 5-H), 3.20, 3.40 (2s, 6H, MeNC(O)NMe).

MS (70 eV): m/z=253.2 (100) [M⁺], 97.1 (92) [M⁺—C₆H₈N₂O₃], 82.1 (80) [M⁺—C₆H₈N₂O₃- Me].

C₁₂H₁₉N₃O₃ (253.1), HRMS: 253.1426.

3-(1,3-Dimethyl-2, 4, 6-troxohexahydropyrimidin-5-yl)-hexahydropyrrolidine (6f)

N-Cbz-Pyrrolidine-2-carbaldehyde (29.14 mg, 125 μmol), N,N-dimethylbarbituric acid (19.5 mg, 125 μmol) and benzyl vinyl ether (134 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals.

¹H-NMR (500 MHz, DMSO-D₆): δ=1.84-1.92, 1.98-2.08. 2.17-2.26, 2.44-2.54 (4m, 6H, 2-H, 6-H, 7-H), 3.04-3.11, 3.20-3.25, 3.37-3.58 (3m, 4H, 3-H, 5-H), 3.22 (s, 6H, NMe), 3.77-3.82 (m, 1H, 7a-H), 4.42-4.60 (m, 1H, 1-H).

³C-NMR (50 MHz, CD₃OD): δ=24.88, 24.64, 30.19, 30.64, 30.97 (C-2, C-6, C-7), 27.74, 27.97, 28.10 28.24 (NMe), 42.36, 42.07 (C-1), 52.94, 55.65, 56.35, (C-3, C-5), 68.63, 68.81, 70.45 (C-7a), 81.34, 84.94, 85.25 (C-5′), 154.7, 154.8 (C-2′), 165.2, 165.3 (C-4′, C-6′).

MS (70 eV): m/z=265.2 (10) [M⁺], 156.1 (45) [C₆H₈N₂O₃], 109.1 (73) [M⁺—C₆H₈N₂0₃].

C₁₃H₁₉N₃O₃ (265.2).

3-(1,3-Dimethyl-2, 4, 6-trioxohexahydropyrimidin-5-yl)-2,4-dimethylpyrrolidine (6g)

N-Cbz-2-Aminopropionaldehyde (25.9 mg, 125 μmol), N,N-dimethylbarbituric acid (19.5 mg, 125 μmol) and benzyl prop-1-enyl ether (148 mg, 1.00 mmol) were reacted with 0.5 ml of toluene, 0.1 ml of trimethyl orthofornate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was 97%.

¹H-NMR (500 MHz, DMSO-D₆): δ=0.74,0.85 (2d, J=7 Hz, 3H, 1′″-H), 1.11, 1.19 (2d, J=7 Hz, 3H, 1″-H), 2.48-2.56 (m, 1H, 4-H), 2.64-2.94 (3m, 2H, 5-H), 3.02, 3.08 (m, 1H, 2-H), 3.30-3.38 (m 1H, 2-H), 3.90-3.98, 4.02-4.10 (2m, 1H, 3-H).

¹³C-NMR (125 MHz, DMSO-D₆): δ=14.33, 16.26, 17.40 (1″-H, 1′″-H), 26.68, 26.94 (NMe), 33.72, 34.00, 44.43, 49.26 (C-3, C-4), 49.79, 50.40 (C-5), 56.10, 57.72 (C-2), 78.62, 81.78 (C-5′), 152.5, 152.8 (C-2′), 162.2, 162.7 (C-4′, C-6′).

MS (70 eV): m/z=253.2 (26) [M⁺], 238.2 (23) [M+- Me], 97.1 (100) [M⁺—C₆H₈N₂O₃], 82.1 (98) [M⁺—C₆H₈N₂O₃ - Me].

C₁₂H₁₉N₃O₃ (253.1), HRMS: 253.1426.

3-(1,3-Dimethyl-2, 4, 6-trioxohexahydropyrimidin-5-yl)-4-ethyl-2-methylpyrrolidine (6h)

N-Cbz-2-Aminopropionaldehyde (25.9 mg, 125 μmol), N,N-dimethylbarbituric acid (19.5 mg, 125 μmol) and benzyl but-1-enyl ether (162 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was 97%.

¹³C-NMR (50 MHz, CD₃OD): δ=12.62, 13.37 (C-2′″), 16.47, 17.90 (C-1″), 23.98, 27.28 (C-1′″), 227.99, 28.06, 28.24, 28.61 (NMe), 42.85, 43.28, 45.18, 48.86 (C-3, C-4), 60.35 (C-5), 58.55, 60.35 (C-2), 154.8, 154.9 (C-2′), 165.4, 165.6, 165.7, 165.8, 166.0 (C-4′, C-6′).

MS (70 eV): m/z=267.2 (8) [M⁺], 252.2,46) [M⁺- Me], 223.2 (1) [M⁺- Me - Et], 111.1 (18) [M⁺—C₆H₈N₂O₃].

C₁₃H₂₁N₃O₃ (267.2), HRMS: 267.1582.

3-(1,3-Dimethyl-2, 4, 6-trioxohexahydropyrimidin-5-yl)-4-isopropyl-2-methylpyrrolidine (6i)

N-Cbz-2-Aminopropionaldehyde (25.9 mg, 125 μmol), N,N-dimethylbarbituric acid (19.5 mg, 125 μmol) and benzyl 3-methylbut-1-enyl ether (190 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was 75%.

¹H-NMR (500 MHz, DMSO-D₆): δ=0.85-1.20 (several d, 9H, J=7-8 Hz, 1″-H, 2′″-H, 3′″-H), 1.30-1.56 (2m, 1H, 1′″-H), 1.85-2.15 (2m, 1H, 4-H), 3.00-3.80 (several m, 10H, 2×NMe, 5-H, 2-H, 3-H).

¹³C-NMR (125 MHz, DMSO-D₆): δ=13.05, 13.46, 16.33, 18.13, 19.21, 20.22, 20.91, 21.49, 21.66, 22.06, 22.50, 22.73, 24.39, 26.77, 26.87, 26.99, 27.19, 27.23, 27.29, 27.45, 28.14, 29.34, 32.08 (-Me, -iPr, NMe), 42.33, 47.71 (C-3, C-4), 48.04, 48.55 (C-5), 68.57, 60.12 (C-2), 83.46, 86.47 (C-5′), 152.5 (C-2′), 162.8, 162.9 (C-4′, C-6′).

MS (70 eV): m/z=281.2 (69) [M⁺], 266.2 (24) [M⁺—Me], 239.1 (86) [M⁺-iPr+H].

C₁₄H₂₃N₃O₃ (281.2), HRMS: 281.1739.

3-(1,3-Dimethyl-2, 4, 6-trioxohexahydropyrimidin)-5-yl)-2-isopropylpyrrolidine (6k)

N-Cbz-2-Amino-3-methylbutyraldehyde (29.4 mg, 125 μmol), N,N-dimethylbarbituric acid (19.5 mg, 125 μmol) and benzyl vinyl ether (134 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was 95%.

¹H-NMR (300 MHz, MeOH-D₄): δ=0.82-1.10 (2d, J=6 Hz, 2″-H, 3″-H), 1.80-2.30 (m, 3H, 4-H, 1″-H), 3.24 (s, 6H, NMe), 3.20-3.58 (2m, 3H, 5-H, 2-H), 3.75-3.82 (m, 1H, 3-H).

MS (70 eV): m/z=267.2 (1) [M^{+],} 224.1 (100) [M⁺- iPr], 111.1 (6) [M⁺—C₆H₈N₂O₃].

C₁₃H₂₁N₃O₃ (267.2).

3-(1,3-Dimethyl-2, 4, 6-trioxohexahydropyrimidin-5-yl)-2-isopropyl-4-methylpyrrolidine (6l)

N-Cbz-2-Amino-3-methylbutyraldehyde (29.4 mg, 125 μmol), N,N-dimethylbarbituric acid (19.5 mg, 125 μmol) and benzyl prop-1-enyl ether (148 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was 78%.

¹³C-NMR (125 MHz, DMSO-D₆): δ=12.90, 14.96, 16.18, 18.96, 19.39, 19.64, 20.28, 20.39, 26.83, 27.31, 27.64, 29.71, 31.14, 34.93, 35.19, 35.48, 39.11, 39.76, 40.66, 45.11 (-Me, -iPr, NMe), 50.33, 50.83, 51.73 (C-5), 65.43, 68.39, 69.44 (C-2), 80.11, 80.51, 82.82 (C-5′), 152.8 (C-2′), 162.9, 163.4, 163.8 (C-4′, C-6′). MS (70 eV): m/z=281.2 (10) [M⁺], 238.1 (100) [M⁺- iPr], 223.1 (1) [M⁺- iPr+Me].

C₁₄H₂₃N₃O₃ (281.2), HRMS: 281.1739.

3-(1,3-Dimethyl-2, 4, 6-trioxohexahydropyrimidin-5-yl)-2,4-diisopropylpyrrolidine (6m)

N-Cbz-2-Amino-3-methylbutyraldehyde (29.4 mg, 125 μmol), N,N-dimethylbarbituric acid (19.5 mg, 125 μmol) and benzyl 3-methylbut-1-enyl ether (190 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was 82%.

¹H-NMR (500 MHz, DMSO-D₆): δ=0.72-0.98 (several d, J=7-8 Hz, 12H, 2″-H, 3″-H, 2′″-H, 3′″-H), 1.30-1.40 (m, 1H, 1′″-H), 1.48-1.82 (2m, 2H, 4-H, 1″H), 3.00-3.12 (m, 8H, 2×NMe, 5-H), 3.32-3.58 (2m, 1H, 2-H), 3.72-3.88 (2m, 1H, 3-H).

¹³C-NMR (125 MHz, DMSO-D₆): δ=19.27, 21.87, 20.06, 20.18, 20.25, 20.40, 20.90, 21.47, 21.71, 22.10, 22.59, 26.75, 27.12, 27.28, 27.32, 27.49, 27.55, 28.02, 29.40, 31.66, 32.14 (2×-iPr, NMe), 39.67, 39.93, 40.10 (C-3, C-4), 48.55, 48.95, 49.90 (C-3, C-4), 49.04, 49.96, 51.90 (C-5), 68.26, 70.01, 70.15 (C-2), 80.26, 84.10, 87.26 (C-5′), 152.4, 152.8 (C-2′), 162.4, 162.5, 163.4, 163.5, 163.7 (C-4′, C-6′).

MS (70 eV): m/z=309.2 (21) [M⁺], 266.2 (100) [M⁺- iPr], 110.1 (25) [M⁺—C₆H₈N₂O₃-iPr].

C₁₆H₂₇N₃O₃ (309.2), HRMS: 309.2052.

3-(1,3-Dimethyl-2, 4, 6-trioxohexalhydlropyrimidin-5-yl)-2-benzylpyrrolidine (6n)

N-Cbz-2-Amino-3-phenylpropionaldehyde (35.4 mg, 125 μmol), N,N-dimethylbarbituric acid (19.5 mg, 125 μmol) and benzyl but-1-enyl ether (162 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was 97%.

¹H-NMR (300 MHz, CD₃OD): δ=2.05-2.45 (2m, 2H, 4-H), 2.90-2.95 (m, 2H, PhCH₂), 3.20 (s, 6H, 2NMe), 3.30-3.55 (m, 3H, 2-H, 5-H), 4.22-4.32 (m, 1H, 3-H), 7.15-7.30 (m, 5H, PhH).

¹³ C-NMR (75 MHz, DMSO-D₆): δ=26.17 (NMe), 27.21 (C-4), 39.87 (C-3), 35.28, 43.49 (C-1″, C-5), 61.83 (C-2), 81.15 (C-5′), 125.5, 128.3, 128.7 (C-3″, C-4″, C-5″, C-6″), 131.0 (C-2″), 152.6 (C-2′), 162.0 (C-4′, C-6′). MS (70 eV): m/z=315.2 (1) [M⁺], 224.1 (100) [M⁺- Bn], 91.0 (10) [Bn].

C₁₅H₂₁N₃O₃ (315.2), HRMS: 315.1582.

3-(1,3-Dimethyl-2, 4, 6-trioxohexahydropyrimidin-5-yl)-2-isobutylpyrrolidine (6o)

N-Cbz-2-Amino-4-methylpentanal (31.2 mg, 125 μmol), N,N-dimethylbarbituric acid (19.5 mg, 125 μmol) and benzyl vinyl ether (134 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was 99%.

¹H-NMR (500 MHz, DMSO-D₆): δ=0.88 (d, J=8 Hz, 6H, 3″-H, 4″-H), 1.44-1.57, 1.60-1.70 (3H, 2″-H, 1″-H), 2.04-2.37 (2m, 2H, 4H), 3.14 (s, 6H, NMe), 3.16-3.47 (2m, 3H, 2-H, 5-H), 3.98-4.03 (m, 1H, 3-H).

¹³C-NMR (50 MHz, CD₃OD): δ=22.38, 23.24, 26.80, 27.99 (C-2″, C-3″, C-4″, NMe), 28.60 (C-4), 41.57 (C-3), 41.97, 45.59 (C-1″, C-5), 60.72 (C-2), 86.10 (C-5′), 154.7 (C-2′), 165.1 (C-4′, C-6′).

C₁₄H₂₃N₃O₃ (281.2), HRMS: 281.1739.

3-(1,3-Dimethyl-2, 4, 6-trioxohexahydropyrimidin-5-yl)-2-isobutyl-4-methylpyrrolidine (6p)

N-Cbz-2-Amino-4-methylpentanal (31.2 mg, 125 μmol), N,N-dimethylbarbituric acid (19.5 mg, 125 μmol) and benzyl prop-1-enyl ether (148 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was>95%.

¹H-NMR (500 MHz, DMSO-D₆): δ=0.75-0.88 (several d, J=7 Hz, 9H, 3″-H, 4″-H, 1′″-H), 1.35-1.65 (3m, 3H, 1″-H, 2″-H), 2.38-2.50 (m, 1H, 4-H), 3.00-3.40 (3m, 3H, 2-H, 5-H), 3.05, 3.08 (2s, 6H, NMe), 3.95-4.10 (2m, 1H, 3-H).

¹³C-NMR (125 MHz, DMSO-D₆): δ=13.79, 16.43 (C-1′″), 20.35, 21.81, 22.32, 22.38, 22.48, 22.90, 22.96, 24.76, 25.01, 25.04, 22.96, 22.99, 27.07, 27.14, 27.20, 27.26 (C-3″, C-4″, C-2″, NMe), 33.94, 34.39, 42.68, 43.02 (C-3, C-4), 40.69, 41.49 (C-1″), 49.99, 51.09, 51.41 (C-5), 59.22, 60.01 (C-2), 79.09, 81.84 (C-5′), 152.9 (C-2′), 162.4 (C-4′, C-6′).

MS (70 eV): m/z=295.2 (11) [M⁺], 238.1 (100) [M^{+- iBu],} 139.1 (30) [M⁺—C₆H₈N₂O₃].

C₁₅H₂₅N₃O₃ (295.2), HRMS: 295.1895.

3-(1,3-Dimethyl-2, 4, 6-trioxohexahydropyrimidin-5-yl)-4-ethyl-2-isobutylpyrrolidine (6q)

N-Cbz-2-Amino-4-methylpentanal (31.2 mg, 125 μmol), N,N-dimethylbarbituric acid (19.5 mg, 125 μmol) and benzyl but-1-enyl ether (162 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was>95%.

¹H-NMR (500 MHz, DMSO-D₆): δ=0.84-0.95 (several d, J=6.5 Hz, 9H, 2′″-H, 3″-H, 4″-H), 1.19-1.72 (5m, 5H, 1″-H, 2″-H, 1′″-H), 2.32-2.42 (m, 1H, 4-H), 3.22-3.26 (several s, 6H, NMe), 3.30-3.43 (2m, 2H, 5-H), 3.48-3.60 (2m, 1H, 2-H), 4.05-4.17 (2m, 1H, 3-H).

¹³C-NMR (50 MHz, CD₃OD): δ=12.62, 13.43 (C-2″), 23.42, 26.93 (C-1′″), 22.76, 22.80, 22.68, 26.76, 27.94, 28.00, 28.45 (C-3″, C-4″, C-2″, NMe), 42.17, 43.15 (C-1″), 42.80, 43.42, 43.76 (C-3, C-4), 50.41, 51.59 (C-5), 60.73, 62.59 (C-2), 86.54 (C-5′), 154.7 (C-2′), 154.4, 165.9 (C-4′, C-6′). MS (70 eV): m/z=309.2 (16) [M⁺], 252.2 (100) [M⁺-iBu].

C₁₆1H₂₇N₃O₃ (309.2), HRMS: 309.2052.

3-(1,3-Dimethyl-2, 4, 6-trioxohexahydropyrimidin-5-yl)-2-isobutyl-4-isopropylpyrrolidine (6r)

N-Cbz-2-Amino-4-methylpentanal (31.2 mg, 125 μmol), N,N-dimethylbarbituric acid (19.5 mg, 125 μmol) and benzyl 3-methyl but-1-enyl ether (190 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals.

¹³C-NMR (75 MHz, DMSO-D₆): δ=21.67, 22.11, 22.44, 22.52, 24.70, 24.88, 26.77, 27.04, 27.25, 27.75 (2×- iPr, NMe), 42.20 (C-1″), 40.78, 48.33, (C-3, C-4), 49.33 (C-5), 59.69, 62.20 (C-2), 83.54 (C-5′), 152.3, 152.6, 152.8 (C-2′), 161.8, 162.5, 162.6 (C-4′, C-6′).

MS (70 eV): m/z=323.2 (22) [M⁺], 266.2 (100) [M⁺- iBu].

C₁₇H₂₉N₃O₃ (323.2), HRMS: 323.2208.

4-(1,3-Dimethyl-2, 4, 6-trioxohexahydropyrimidin-5-yl)-azepane (8a)

N-Cbz-Pyrrolidin-2-ol (27.6 mg, 125 μmol), N,N-dimethylbarbituric acid (19.5 mg, 125 μmol) and benzyl vinyl ether (134 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was 98%.

¹³C-NMR (125 MHz, DMSO): δ=22.80, 23.54, 29.89, 29.93 (C-3, C-4, C-6), 26.82, 26.88, 26.90, 27.04, 32.00 (NMe, C-5), 46.54, 46.60 (C-2, C-7), 89.50 (C-5′), 152.5 (C-2′), 162.1 (C-4′), 162.1 (C-4′, C-6′).

C₁₂H₁₉N₃O₃ (253.2).

4-(1,3-Dimethyl-2, 4, 6-trioxohexahydropyrimidin-5-yl)-3-methylazepane (8b)

N-Cbz-Pyrrolidin-2-ol (27.6 mg, 125 μmol), N,N-dimethylbarbituric acid (19.5 mg, 125 μmol) and benzyl prop-1-enyl ether (148 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was>95%.

¹³C-NMR (125 MHz, DMSO): δ=21.15, 23.16, 26.96, 27.23, 27.44, 32.89 (C-3, C-4, C-5, C-6, C-1″, NMe), 48.64, 53.30 (C-2, C-7), 88.14 (C-5′), 152.4 (C-2′), 162.5 (C-4′, C-6′).

C₁₃H₂₁N₃O₃ (267.2).

3-(2,4-Dioxochroman-3-yl)-2-methylpyrrolidine (6s)

N-Cbz-2-Aminopropionaldehyde (25.9 mg, 125 μmol), 4-hydroxycoumarin (20.25 mg, 125 μmol) and benzyl vinyl ether (134 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was 95.0%.

¹H-NMR (500 MHz, DMSO-D₆): δ=1.10, 1.14 (2d, J=6 Hz, 3H, 1″-H), 2.00-2.20 (2m, 2H, 4-H), 3.15-3.24, 3.30-3.45 (2m, 3H, 2-H, 5-H), 3.72-3.80, 3.88-3.95 (2m, 1H, 3-H), 7.02-7.14 (m, 2H, 6′-H, 7′-H), 7.30-7.41, 7.74-7.82 (2m, 2H, 5′-H, 8′-H).

C₁₄H₁₅NO₃ (245.1), HRMS: 245.1051.

4-(2,4-Dioxochroman-3-yl)piperidine (7h)

N-Cbz-3-Aminopropionaldehyde (25.9 mg, 125 μmol), 4-hydroxycoumarin (20.25 mg, 125 μmol) and benzyl vinyl ether (134 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was>95%.

¹³C-NMR (125 MHz, DMSO-D₆): δ=25.65 (C-3, C-5), 30.80 (C-4), 44.25 (C-2, C-6), 102.9 (C-3′), 115.7, 122.7, 124.4, 130.7 (C-5′, C-6′, C-7′, C-8′), 120.0 (C-4a), 152.9 (C-8a), 162.9, 166.7 (C-2′, C-4′).

C₁₄H₁₅NO₃ (245.1), HRMS: 245.1051.

3-(3,3-Dimethyl-2,6-dioxocyclohexyl)-2-methylpyrrolidine (6t)

N-Cbz-2-Aminopropionaldehyde (25.9 mg, 125 μmol), 4,4-dimethylcyclohexane-1,3-dione (17.5 mg, 125 μmol) and benzyl vinyl ether (134 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was>95%.

¹H-NMR (300 MHz, DMSO-D₆): δ=0.92 (s, 6H, C(CH₃)₂), 1.00-1.05 (m, 3H, 1″-H), 1.57 (t, J=5.5 Hz, 2H, 5′-H), 1.62-2.05 (m, 2H, 4-H), 2.15 (t, J=5.5 Hz, 2H, 6′-H), 3.00-3.42 (3m, 3H, 2-H, 5-H), 3.60 (m, 1H, 3-H).

C₁₃H₂₁NO₂ (223.2).

3-(4,4-Dimethyl-2,6-dioxocyclohexyl)-2-methylpyrrolidine (6u)

N-Cbz-2-Aminopropionaldehyde (25.9 mg, 125 μmol), 5,5-dimethylcyclohexane-1,3-dione (17.5 mg, 125 μmol) and benzyl vinyl ether (134 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was>95%.

¹H-NMR (300 MHz, DMSO-D₆): δ=0.86, 0.87 (2s, 6H, C(CH₃)₂), 0.95, 1.05 (2d, J=6 Hz, 3H, 1″-H), 1.78-2.22 (m, 2H, 4-H), 1.96, 1.97 (2s, 4H, 3′-H, 5′-H), 2.78-3.30 (m, 3H, 2-H, 5-H), 3.66-3.80 (m, 1H, 3-H).

C₁₃H₂₁NO₂ (223.2).

4-(1,3-Dimethyl-2, 4, 6-trioxohexahydropyrimidin-5-yl)-piperidine (7a)

N-Cbz-3-Aminopropionaldehyde (25.9 mg, 125 μmol), N,N-dimethylbarbituric acid (19.5 mg, 125 μmol) and benzyl vinyl ether (134 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was>95%.

¹³C-NMR (50 MHz, CD₃OD): δ=22.55 (C-3, C-5), 28.02 (NMe), 32.74 (C-4), 46.53 (C-2, C-6), 91.72 (C-5′), 154.7 (C-2′), 165.1 (C-4′, C-6′).

MS (70 eV): m/z=239.2 (37) [M⁺], 156.1 (48) [C₆H₈N₂O₃], 83.0 (100)[M⁺—C₆H₈N₂O₃].

C₁₁H₁₇N₃O₃ (239.2).

4-(1,3-Dimethyl-2, 4, 6-trioxohexahydropyrimidin-5-yl)-5-methlylpiperidine (7b)

N-Cbz-3-Aminopropionaldehyde (25.9 mg, 125 μmol), N,N-dimethylbarbituric acid (19.5 mg, 125 μmol) and benzyl prop-1-enyl ether (148 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals.

MS (70 eV): m/z=253.2 (18) [M⁺], 238.1 (5) [M⁺-Me], 98.1 (100) [M⁺—C₆H₈N₂O₃].

C₁₂H₁₉N₃O₃ (253.1), HRMS: 253.1426.

4-(1,3-Dimethyl-2, 4, 6-trioxohexahydropyrimidin-5-yl)-5-ethylpiperidine (7c)

N-Cbz-3-Aminopropionaldehyde (25.9 mg, 125 μmol), N,N-dimethylbarbituric acid (19.5 mg, 125 μmol) and benzyl but-1-enyl ether (162 mg, 1.00 mmol) were reacted with 0.5 ml of toluene, 0.1 ml of trimethyl orthoformnate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals.

MS (70 eV): m/z=267.1 (30), 238.1 (18) [M^{+- Et],} 156.1 (38) [C₆H₈N₂O₃], 112.1 (89) [M⁺—C₆H₈N₂O₃].

C₁₃H₂₁N₃O₃ (267.2), HRMS: 267.1582.

4-(1,3-Dimethlyl-2, 4, 6-trioxohexahydropyrimidin-5-yl)-5-isopropylpiperidine (7d)

N-Cbz-3-Aminopropionaldehyde (25.9 mg, 125 μmol), N,N-dimethylbarbituric acid (19.5 mg, 125 μmol) and benzyl 3-methyl but-1-enyl ether (190 mg, 1.00 mmol) were reacted with 0.5 ml of-toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals.

MS (70 eV): m/z=281.1 (33) [M⁺], 238.1 (20) [M⁺- iPr], 156.0 (100) [C₆H₈N₂O₃], 126.1 (55) [M⁺—C₆H₈N₂O₃].

C₁₄H₂₃N₃O₃ (281.2), HRMS: 281.1817.

4-(1,3-Dimethyl-2, 4, 6-trioxohexahydropyrimidin-5-yl)-2,5-dimethylpiperidine (7e)

N-Cbz-3-Benzylaminobutyraldehyde (38.9 mg, 125 μmol), N,N-dimethylbarbituric acid (19.5 mg, 125 μmol) and benzyl prop-1-enyl ether (148 mg, 1.00 mmol) were reacted with 0.5 ml of toluene, 0.1 ml of trimethyl orthoformate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was>95%.

MS (70 eV): m/z=267.1 (30) [M⁺], 252.2 (33) [M⁺- Me], 156.1 (7) [C₆H₈N₂O₃], 112.1 (100) [M^{+-C}₆H₈N₂O₃], 96.1(65) [M⁺—C₆H₈N₂O₃- Me].

C₁₃H₂₁N₃O₃ (267.2).

1-Butyl-4-(1,3-dimethyl-2, 4, 6-trioxohexahydropyrimidin-5-yl)-2-methylpiperidine (7f)

N-Cbz-3-Butylaminobutyraldehyde (34.7 mg, 125 μmol), N,N-dimethylbarbituric acid (19.5 mg, 125 μmol) and benzyl vinyl ether (134 mg, 1.00 mmol) were reacted with 0.5 ml of toluene, 0.1 ml of trimethyl orthoformate and a few crystals of ethylenedianunonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals. The purity determined by HPLC was>95%.

MS (70 eV): m/z=309.3 (7) [M⁺], 294.3 (43) [M⁺- Me], 266.7 (100) [M⁺—C₃H₇], 138.2 [M⁺—C₆H₈N₂O₃ - Me].

C₁₆H₂₇N₃O₃ (309.2), HRMS: 309.2052.

1-Butyl-4-(1,3-dimethyl-2, 4, 6-trioxohexahydropyrimidin-5-yl)-2,5-dimethylpiperidine (7g)

N-Cbz-3-Butylaminobutyraldehyde (34.7 mg, 125 μmol), N,N-dimethylbarbituric acid (19.5 mg, 125 μmol) and benzyl prop-1-enyl ether (148 mg, 1.00 mmol) were reacted with 0.5 ml of toluene, 0.1 ml of trimethyl orthofornate and a few crystals of ethylenediammonium diacetate (EDDA) under an argon atmosphere in a 10 ml pressurized flask in accordance with the GOP. The product, after dissolution in methanol and subsequent removal of the solvent, was obtained as a yellowish oil which later crystallized out as amorphous crystals.

MS (70 eV): m/z=323.2 (8) [M⁺], 280.2 (100) [M⁺ C₃H₇].

C₁₆H₂₇N₃O₃ (323.2), HRMS: 323.2208.

1. A process for the combinatorial preparation of a substance library from n different compounds in an array of n different reaction solutions at n spatially separate reaction sites, in which at each of the n reaction sites
(a) from in each case m reactants, in one reaction or reaction sequence, a zwitterionic compound is obtained dissolved in a polar solvent,
(b) the zwitterionic compound is precipitated as a solid by addition of a nonpolar solvent, isolated and then obtained in pure form,
(c) if appropriate the zwitterionic compound is further reacted, with each of the n reaction solutions differing from each of the n−1 other reaction solutions in at least one of the m reactants.

2. The process as claimed in claim 1, wherein, as the m reactants, a protected aminoaldehyde of the general formula 1, 2 or 3 is reacted with an enol ether of the general formula 5 and a 1,3-dicarbonyl compound 4 in the reaction sequence A below to give a betaine of the general formula 6, 7 or 8 as zwitterionic compound. 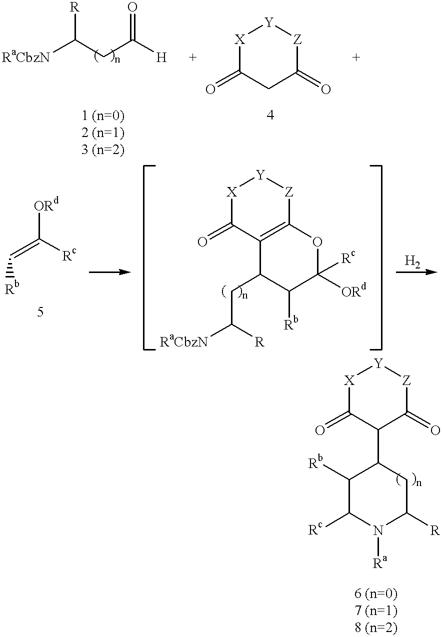
where R^{d}=benzyl or Si(CH₃)₃.

3. The process as claimed in claim 2, wherein the protected aminoaldehyde is selected from the alpha-, beta- and gamma-aminoaldehydes of the general formulae 1a to 1e: 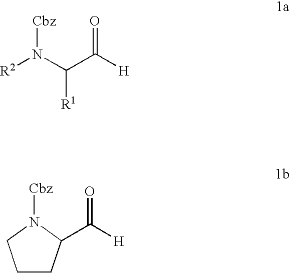
where
R¹=H, alkyl, CH₂OH, CH₂SH,
R²=H, alkyl and
alkyl=Me, Et, n-Pr, iPr, n-Bu, sec-Bu, iBu or pentyl 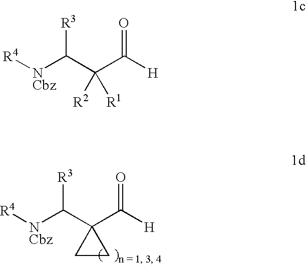
where
R¹, R² are identical or different and are H, alkyl
R³, R⁴ are identical or different and are H, alkyl,
and alkyl=Me, Et, n-Pr, iPr, n-Bu, sec-Bu, iBu or pentyl 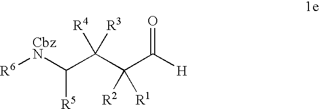
where
R¹, R², R³, R⁴ are identical or different and are H, alkyl
R⁵, R⁶ are identical or different and are H, alkyl,
and alkyl Me, Et, n-Pr, n-Bu, sec-Bu, iBu or pentyl.

4. The process as claimed in claim 2, wherein the enol ether is selected from the enol ethers of the general formulae 2a to 2d: 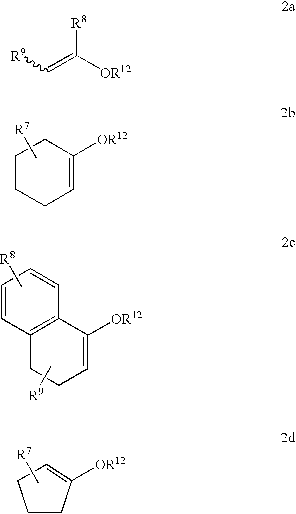
where
R⁷, R⁸, R⁹ are identical or different and are H, alkyl, Bn, Ph-C₂H₄, CH₂OR, CH₂SR, CH₂NHAc, CH₂CH₂OR, CH₂CH₂SR, CH₂CH₂NHAc and
R⁹ is additionally Ph,
R¹² benzyl or Si(CH₃)₃,
and alkyl=Me, Et, n-Pr, iPr, n-Bu, sec-Bu, iBu or pentyl.

5. The process as claimed in claim 2, wherein the 1,3-dicarbonyl compound is selected from the 1,3-dicarbonyl compounds of the general formulae 3a and 3b: 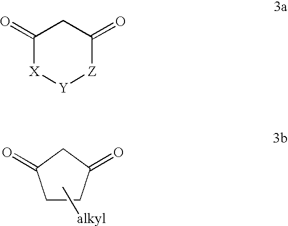
where
X, Z, Y are identical or different and are NH, N-alkyl, CH₂, CMe₂, O, S and
Y CH₂, CMe₂, C═O, C═S, or
X—Y—Z is NH—CR¹⁰═CR¹¹, N-alkyl-CR¹⁰═CR¹¹ O—CR¹⁰O═CR¹¹, N-alkyl-CHR¹-CHR² where R¹⁰ and R¹¹ are identical or different and are alkyl, unsubstituted or with OH, O-alkyl, NH₂, NHalkyl or N(alkyl)₂ substituted phenyl, unsubstituted or with OH, O-alkyl, NH₂, Nhalkyl or N(alkyl)₂ substituted 1,3-butadiene-1,3-dienyl, where in the last case R¹⁰ and R¹¹ are part of the same ring system, and alkyl=Me, Et, n-Pr, iPr, n-Bu, sec-Bu, iBu or pentyl.

6. The process as claimed in claim 1, wherein the zwitterionic compound is produced in water, methanol or their mixtures as polar solvent.

7. The process as claimed in claim 1, wherein the zwitterionic compound is precipitated by adding diethyl ether as nonpolar solvent.
